(19) United States
(12) Patent Application Publication
(10) Pub. No.: US 2014/0272766 A1
(43) Pub. Date: Sep. 18, 2014

(54) HEAD MOUNTED DISPLAY FOR AN INTRA-ORAL IMAGING SYSTEM

(71) Applicant: Ormco Corporation, Orange, CA (US)

(72) Inventors: Craig A. ANDREIKO, (US); Robert F. DILLON, Bedford, NH (US); James A. SICKLES, Round Hill, VA (US)

(73) Assignee: Ormco Corporation, Orange, CA (US)

(21) Appl. No.: 14/209,617

(22) Filed: Mar. 13, 2014

Related U.S. Application Data
(60) Provisional application No. 61/784,498, filed on Mar. 14, 2013.

(51) Int. Cl. A61B 1/00; A61B 1/045; A61B 1/247

(52) U.S. Cl. CPC A61B 1/00048; A61B 1/247; A61B 1/045
USPC 433/27; 433/29

(57) ABSTRACT

Provided is a head mounted display system that communicates with an intra-oral imaging system. Data received from the intra-oral imaging system is displayed in at least one head mounted display unit of the head mounted display system. Provided also is an intra-oral imaging system that in communication with a head mounted display system. The intra-oral imaging system sends data corresponding to an image of at least one tooth acquired by an intra-oral imaging sensor, to be displayed on the head mounted display system. The intra-oral imaging system receives, from the head mounted display system, a selection of at least one operation that is to be performed by the intra-oral imaging system.

### FIELD

The disclosure relates to a system, method, and computer readable storage medium for a head mounted display for an intra-oral imaging system.

### BACKGROUND

A head mounted display system is a device that may be worn on the head. The head mounted display system may include one or more display units to display images or data, where the one or more display units are located in front of the eyes of the wearer and may be embedded in a helmet, an eye-glass, a visor, etc. The display units may comprise video display units that may include Liquid Crystal Displays (LCD), Light Emitting Diode (LED) displays, Optical Light Emitting Diode (OLED) displays, Cathode Ray Tube (CRT) displays, and other types of displays. If only one display unit is located in the head mounted display system the head mounted device may be referred to as a monocular head mounted display system. If two display units are located in the head mounted display system the head mounted display system may be referred to as a binocular head mounted display system.

An intra-oral imaging system is a diagnostic equipment that allows a dental practitioner to see the inside of a patient's mouth and display the topographical characteristics of teeth on a display unit. Certain three-dimensional (3D) intra-oral imagers may be comprised of an intra-oral camera with a light source. The 3D intra-oral imager may be inserted into the oral cavity of a patient by a dental practitioner. After insertion of the intra-oral imager into the oral cavity, the dental practitioner may capture images of visible parts of the teeth and the gingivae. The 3D intra-oral imager may be fabricated in the form of a slender rod that is referred to as a wand or a handpiece. The wand may be approximately the size of a dental mirror with a handle that is used in dentistry. The wand may have a built-in light source and a video camera that may achieve an imaging magnification, ranging in scale from 1/10 to 40 times or more. This allows the dental practitioner to discover certain types of details and defects of the teeth and. gums. The images captured by the intra-oral camera may be displayed on a display unit.

### SUMMARY OF THE PREFERRED EMBODIMENTS

Provided are a system, method, and computer readable storage medium in which a head mounted display system communicates with an intra-oral imaging system. Data received from the intra-oral imaging system is displayed in at least one head mounted display unit of the head mounted display system.

In certain embodiments, data obtained from a measurement device is processed to determine movement of a head of a wearer of the head mounted display unit. Based on the determined head movement, at least one operation performed by the intra-oral imaging system is selected.

In certain additional embodiments, the measurement device is selected from a group consisting of an accelerometer and a global positioning system receiver.

In further embodiments, the at least one head mounted display unit is selected from a plurality of head mounted display units, wherein the plurality of head mounted display units is comprised of a first head mounted display unit and a second head mounted display unit. The first head mounted display unit displays a surface imagery of a tooth received from the intra-oral imaging system to a left eye of a wearer of the head mounted display system. The second head mounted display unit displays the surface imagery of the tooth received from the intra-oral imaging system to a right eye of the wearer of the head mounted display system.

In yet further embodiments, the head mounted display system receives control instructions from a wand of the intra oral imaging system. The at least one head mounted display unit displays an image acquisition window displayed in a display unit included in the intra-oral imaging system.

In certain embodiments, the at least one head mounted display unit is a see-through display unit that allows a patient's environment to be viewed simultaneously with the data acquired via the intra-oral imaging system.

In further embodiments, the displayed data corresponds to an image received from the intra-oral imaging system. A contrast setting is regulated to modify a contrast of the image received from the intra-oral imaging system. Power is provided to the intra-oral imaging system via a battery.

Provided also is system that is in communication with a head mounted display system, where the system comprises an intra-oral imaging sensor, and a processor coupled to the intra-oral imaging sensor. The processor performs operations to send data corresponding to an image of at least one tooth acquired by the intra-oral imaging sensor to be displayed on the head mounted display system. The processor receives, from the head mounted display system, a selection of at least one operation that is to be performed by the processor.

### BRIEF DESCRIPTION OF THE DRAWINGS

Referring now to the drawings in which like reference numbers represent corresponding parts throughout:

FIG. 1 illustrates a block diagram of a computing and imaging environment that includes a head mounted display system in communication with an intra-oral imaging system, in accordance with certain embodiments;

FIG. 2 illustrates a diagram in which an exemplary head mounted display system is used to view images of teeth being acquired via an intra-oral imaging system, in accordance with certain embodiments;

FIG. 3 illustrates an exemplary intra-oral imaging system with which the head mounted display system is in communication, in accordance with certain embodiments;

FIG. 4 illustrates controls in a wand of the intra-oral imaging system, where the controls are used to control certain operations of the head mounted display system, in accordance with certain embodiments;

FIG. 5 illustrates a first flowchart that shows certain operations performed at least by the head mounted display system, in accordance with certain embodiments;

FIG. 6 illustrates a second flowchart that shows operations performed by the head mounted display system, in accordance with certain embodiments;

FIG. 7 illustrates a third flowchart that shows operations performed by the infra-oral imaging system, in accordance with certain embodiments; and

FIG. 8 illustrates a block diagram of a computational device that shows certain elements of the head mounted display or the intra-oral imaging device shown in FIG. 1, in accordance with certain embodiments.

### DETAILED DESCRIPTION

In the following description, reference is made to the accompanying drawings which form a part hereof and which illustrate several embodiments. It is understood that other embodiments may be utilized and structural and operational changes may be made.

Certain embodiments integrate a head mounted display system with an intra-oral imaging system. The head mounted display system displays images acquired by the intra-oral imaging system. The head mounted display system also displays data and information generated by the intra-oral imaging system.

In certain embodiments, the movements of the head of the wearer of the head mounted display system may be used to control certain operations of the intra-oral imaging system. In certain additional embodiments, controls on the wand of the intra-oral imaging system may be used to control certain operations of the head mounted display system.

The displays located in the head mounted display system is closer to the wearer of the head mounted display system in comparison to relatively large conventional displays such as touch screen, CRT displays, etc., that are coupled to the intra-oral imaging system. Additionally, the displays located in the head mounted display system allow the wearer to view images, textual material, graphical user interfaces, and other data at a better angle in comparison to large conventional displays such as touch screen, CRT display, etc., that are coupled to the intra-oral imaging system. As a result, operators may find it preferable to use the head mounted display system in association with the intra-oral imaging system, rather than use the intra-oral imaging system without the head mounted display system.

### EXEMPLARY EMBODIMENTS

FIG. 1 illustrates a block diagram of a computing and imaging environment 100 that includes ahead mounted display system 102 in communication with an intra-oral imaging system 104, in accordance with certain embodiments.

The head mounted display system 102 may comprise any suitable head mounted display system known in the art, and may be worn on the head of a wearer, such as a robot 106 or a human operator 108.

The head mounted display system 102 may include one or more head mounted display units 110, 112 that display images, data, graphical user interfaces, texts, etc. The head mounted display units 110, 112 are located in front of the eyes of the wearer 106, 108 and may be embedded in a helmet, an eye-glass, a visor, etc. The head mounted display units 110, 112 may comprise any suitable video display unit and may include Liquid Crystal Displays (LCD), Light Emitting Diode (LED) displays, Optical Light Emitting Diode (OLD) displays, Cathode Ray Tube (CRT) displays, and other types of displays. In certain embodiments, at least one of the head mounted display units 110, 112 is a see-through display unit that allows the environment of the operator 106, 108 or a patient to be viewed simultaneously with data acquired via the intra-oral imaging system 104 that is also displayed on the head mounted display units 110, 112.

The head mounted display system 102 also includes a processor 114, a display and control application 116, a measurement device 118, a contrast setting 120, and a battery and recharging unit 122. In certain embodiments, the display and control application 116 may be implemented in software, firmware, hardware or any combination thereof In certain embodiments instructions that comprise the display and control application 116 may execute on the processor 114.

The measurement device 118 of the head mounted display system 102 may include an accelerometer and/or a Global Positioning System (GPS) receiver. The accelerometer may be used to calculate the acceleration of the head mounted display system 102, in response to head movements of the wearer of the head mounted display system 102. The GPS receiver may calculate the three-dimensional position of the head mounted display system 102 via signals received from GPS satellites.

The contrast setting 120 of the head mounted display system 102, may comprise a physical element (e.g., a knob, a switch, etc.) that may be adjusted to control the contrast of the images, texts, graphical user interfaces, and other visual elements displayed on the head mounted display units 110, 112. In certain embodiments, the contrast setting 120 may be adjusted via head movements of the wearer in response to items displayed on the head mounted display units 110, 112. For example, the head mounted display units 110, 112 may indicate via a textual display to the wearer that to increase the contrast the wearer should tilt his or her head up. In response, to the tilting of the head up, the head mounted display system 102 may increase the contrast of the items displayed on the head mounted display units 110, 112.

The battery and recharging unit 122 may include a battery and a recharging unit that recharges the battery. In certain alternative embodiments, the battery may be a disposable battery in which case the recharging unit may be not present. In certain alternative embodiments, the head mounted display system 102 may be directly powered via electricity drawn from power lines via an electrical cord, and in such cases the battery may not be needed for providing power to the head mounted display system 102.

The head mounted display system 102 communicates with the intra-oral imaging system 104 via a wired connection, via a wireless connection, or via any combination thereof, as shown via reference numeral 124. In certain embodiments, the head mounted display system 102 may communicate with the intra-oral imaging system 104 via a network, such as the Internet, an intranet, a local area network, a wide area network, a peer to peer network, etc. In other embodiments, the communication between the head mounted display system 102 and the intra-oral imaging system may be direct, i.e., without the use of a network.

The intra-oral imaging system 104 is comprised of a processor 126, a display unit 128, a wand 130, and an intra-oral imaging application 132. In certain embodiments, the intra-oral imaging system 104 and/or the head mounted display system 102 may be coupled via a wired or wireless connection over a network to one or more computational devices (not shown), where the computational devices may include any suitable computational device such as a personal computer, a server computer, a mini computer, a mainframe computer, a blade computer, a tablet computer, a touch screen computing device, a telephony device, a cell phone, a mobile computational device, etc., and some of the computational devices may provide web services or cloud computing services.

A dental practitioner may hold the wand 130 inside a patient's oral cavity. An optical source coupled to the wand 130 may illuminate the oral cavity and the intra-oral imaging sensor 134 may be used to capture a plurality of digital images of structures in the oral cavity, such as the patient's teeth, gingivae, and/or palate, and other structures, such as fillings, braces, etc. In certain embodiments the intra-oral imaging sensor 134 may comprise an intra-oral camera.

The operation of the wand 130 may be controlled by the intra-oral imaging application 132 that may be implemented in software, hardware, firmware or any combination thereof. In certain embodiments, the display unit 128 of the intra-oral imaging system 104 may include a touch screen display unit that displays an image acquisition window 136 and other windows 138. The intra-oral imaging application 132 may process the images acquired by the intra-oral imaging sensor 134 and display the images on the image acquisition window 136 of the display unit 128 and transmit the images to the head mounted display system 102 for display On the head mounted display units 110, 112. In certain alternative embodiments, the intra-oral imaging application 132 may transmit the data displayed in one or more of the other windows 138 to the head mounted display system 102 for display on the head mounted display units 110, 112. The data displayed in the one or more other windows 138 may include selectable commands used to control the operations of the intra-oral imaging system 104. For example a selectable command may include a command that requests the operator to indicate whether three dimensional surface reconstructions of the patient's teeth should be performed on images acquired via the intra-oral imaging sensor 134.

In addition to the intra-oral imaging sensor 134, the wand 130 may also include one or more physical controls (such as a switch, a knob, etc.) 140 that may be operated by an operator 106, 108 of the wand 130 to control certain operations of the head mounted display system 102 or to control communications with the head mounted display system 102. For example, the operator 106, 108 may press the physical control 140 to the right to start sending the contents image acquisition window 136 to the head mounted display system 102, and may press the physical control 140 to the left to start sending the contents of some other window 138 to the head mounted display system 102.

Therefore, FIG. 1 illustrates certain embodiments in which images and other information displayed on the display unit 128 of an intra-oral imaging system 104 is displayed on a head mounted display system 102 that is worn on the head of a robot 106 or a human operator 108.

FIG. 2 illustrates an embodiment 200 in which an exemplary head mounted display system 102 is used to view images of teeth being acquired via an intra-oral imaging system 104, in accordance with certain embodiments.

FIG. 2 shows that the head mounted imaging system 102 is worn on the head 204 of the human operator 108. The human operator 108 is shown holding the wand 130 of the intra-oral imaging system 104 and acquiring images of an impression of the teeth 206 of a patient. In alternative embodiments, the human operator 108 may use the wand 130 to directly capture images of teeth from the oral cavity of a patient.

The images acquired by the wand 130 of the intra-oral imaging system 104 or three dimensional surfaces constructed from such images, may be displayed in the head mounted imaging system 102 for the eyes of the human operator 108.

FIG. 3 illustrates a view 300 of an exemplary infra-oral imaging system 104 with which the head mounted display system 102 is in communication, in accordance with certain embodiments. The wand 130 having the intra-oral imaging sensor 134 is included in the intra-oral imaging system 104, in accordance with certain embodiments. It should be noted that intra-oral imaging system 104 is exemplary and other intra-oral imaging systems may be used in alternative embodiments.

The intra-oral imaging system 104 may include the wand 130 having the intra-oral imaging sensor 134. The wand 130 is small and light weight for use by dental practitioners, and the intra-oral imaging process is fast and relatively simple to use, allowing the imaging of both arches and bites to be accomplished rapidly, such that a digital model of the imaged areas may be viewed on the display unit 128, where in certain embodiments the display unit 128 is a touch screen display.

The intra-oral imaging system 104 may include a wand storage area 302 in which the wand 130 may be stored. The wand 130 may be extensibly coupled via a cord 304 to the housing 306 of the intra-oral imaging system 104.

The intra-oral imaging system 104 may include a handle 308 that may be used for carrying the intra-oral imaging system 104 from one location to another. The handle 308 may also be referred to as a carrying handle.

In addition to the handle 308, the display unit 128, the wand 130, and the housing 306, the intra-oral imaging system 104 includes a power button 312 that is located on the front face of the intra-oral imaging system 104. The power button 312 may be used to switch the intra-oral imaging system 104 on and off. Additionally, light emitting diode (LED) based indicators 310 may indicate one or more status related to the operational state of the intra-oral imaging system 104.

Therefore, FIG. 3 illustrates certain embodiments in which an intra-oral imaging system 104 includes a wand 130 that includes an intra-oral imaging sensor 134, wherein the intra-oral imaging system 104 communicates images, data, textual material, graphical user interfaces, etc., to the head mounted display system 102.

FIG. 4 illustrates a diagram 400 of a view 402 of an exemplary wand 130 of the intra-oral imaging system 104, where one or more controls 140 are used to control certain operations of the head mounted display system 102, in accordance with certain embodiments. The wand 130 that includes the intra-oral imaging sensor 134 for capturing images for a patient's teeth is shown is FIG. 4, in accordance with certain embodiments. Components of the wand 134 may be wholly or partially enclosed within a housing 402 that protects the optical components of the wand 130 from dust and debris to maintain measurement accuracy.

The tip 404 is the portion of the wand 130 that is inserted into a patient's mouth. The intra-oral imaging sensor 134 may be embedded within the tip 404 of the wand 130. The tip 404 of the wand 130 may include an optical window made of biocompatible, transparent material that may be either plastic or glass. The optical window may be mounted into the plastic tip housing such that no sharp corners or edges contact human tissue. The light from the optical source is transmitted through the optical window, and the intra-oral imaging sensor 134 captures images of the structures of the oral cavity through the optical window. It should be emphasized that the wand tip 404 is designed to be long enough to reach the back teeth of a typical patient.

The wand 130 has a molded area 408 in which there are keypad buttons and controls to traverse through items from the graphical user interfaces and other elements displayed on either the display unit 128 of the intra-oral imaging system 104 or the head mounted display units 110, 112, and for controlling various elements of the wand 130, such as the intra-oral imaging sensor 134. In certain embodiments, the tip 404 of the wand 130 is covered with a disposable molded plastic sheath that snaps on and off the wand 130. The disposable molded plastic sheath may be transparent and may have a mirror.

The end comprising the tip 404 of the wand 130 may be referred to as the distal end 412 of the wand and the end to which the cord 304 is extensibly coupled may be referred to as the proximal end 414 of the wand 130.

Therefore, FIG. 4 illustrates certain embodiments in which a wand 130 includes at least an intra-oral imaging sensor 134 and controls 140 for controlling certain operations of the head mounted display system 102. Additionally, by pressing keypad buttons and controls in the molded area 408, graphical user interfaces and other elements may be traversed in the head mounted display unit 110, 112.

FIG. 5 illustrates a first flowchart 500 that shows certain operations performed at least by the head mounted display system 102, in accordance with certain embodiments.

Control starts at block 502, where an operator, such as the robot 106 or the human operator 108, wears and activates the head mounted display system 102. Control proceeds to block 504 where the operator 106, 108 uses the wand 130 of the intra-oral imaging system 104 inside a patient's oral cavity to acquire intra-oral images of the patient's teeth.

From block 504, control proceeds in parallel to block 506 and block 508. At block 506, the intra-oral images are displayed on the display unit 128 of the intra-oral imaging system, and at block 508 the intra-oral images are displayed on the display unit(s) 110, 112 of the head mounted display system 102.

From block 506 control proceeds to block 510 where the intra-oral images are processed by the intra-oral imaging system 104 to generate surface imagery. The surface imagery provides a three dimensional view of the teeth of the patient. The surface imagery is displayed (at block 512) on the display unit(s) 110, 112 of the head mounted display system 102.

To generate the surface imagery (at block 510) point clouds may processed by the intra-oral imaging application 132 executing in the intra-oral imaging system 104. Aggregated surface samples of the crowns of a patient's teeth acquired by the intra-oral imaging system 104 may be referred to as a point cloud. The crown of the tooth imaged by the intra-oral imaging sensor 134 is a solid object, and the surfaces of the crown correspond to the boundaries of the solid object. The crown surface may be represented by a surface mesh of node points connected by triangles, quadrilaterals or via different types of polygon meshes. In alternative embodiments, a solid mesh may also be used to represent the crown surface. The process of creating the mesh is referred to as tessellation. In certain embodiments, the surface corresponding to the crown is represented in three dimensional space via limited length vectors or via voxels or via other data structures. The voxels may correspond to three-dimensional points on the surface of a crown. In certain embodiments, the limited length vectors may be converted to voxel representation via appropriate three dimensional coordinate transformations. The limited length vectors may correspond to the sides of the different types of polygon meshes (e.g., triangles, quadrilaterals, etc.) in the surface representation of the crown. Other representations of surface imagery may also be used in alternative embodiments.

From block 510, control proceeds to block 514, where the intra-oral imaging system 104 displays a question for the operator 106, 108 and requests a yes or no answer. In alternative embodiments, other types of selectable elements, such as selecting one of multiple choices may be displayed to the operator 106, 108. The question requesting a yes or no answer is transmitted by the intra-oral imaging system 104 to the head mounted display system 102. The question requesting the yes or no answer is received and displayed (at block 516) on the display unit(s) 110, 112 of the head mounted display system 102.

In response to the display of question regarding the yes or no answer on the display units 110, 112 of the head mounted display system 102. the operator 106, 108 nods or shakes his or her head to convey an answer to the question. For example, the nodding of the head may indicate a response of “yes” and the shaking of the head may indicate a response of “no”.

The display and control application 116 that executes in the head mounted display system 102 uses the accelerometer or GPS receiver readings that form the measurement device 118, to determine (at block 520) whether the operator has nodded or shaken his or her head.

Control proceeds to block 522 from block 520, and at block 522 the yes or no answer is determined from the nodding or shaking of head, and appropriate operations are performed (at block 522) by the intra-oral imaging system 104.

Therefore, FIG. 5 illustrates certain embodiments in which images, three-dimensional surfaces, and other data are displayed both on the display unit 128 of the intra-oral imaging system 104 and on the display units 110, 112 of the head mounted display system 102. In certain alternative embodiments, certain information may be displayed only on the head mounted display units 110, 112 and not on the display unit 128 of the intra-oral imaging system. In certain other alternative embodiments, certain information may be displayed only on the display unit 128 of the intra-oral imaging system and not on the head mounted display units 110, 112. In certain embodiments, the intra-oral imaging system may be implemented with no display unit 128 and may rely on the display units 110, 112 of the head mounted display system 102 for displaying information to the operators 106 108.

FIG. 6 illustrates a second flowchart 600 that shows operations performed by the head mounted display system 102, in accordance with certain embodiments. The operations performed by the head mounted display system 102 may be caused by the display and control application 116 executing on the processor 114 of the head mounted display system 102.

Control starts at block 602, in which the head mounted display system 102 communicates with the intra-oral imaging system 104. Data received from the intra-oral imaging system is displayed (at block 604) in at least one head mounted display unit (e.g., display unit 110 or 112) of the head mounted display system 102.

FIG. 7 illustrates a third flowchart 700 that shows operations performed by the intra oral imaging system 104 in accordance with certain embodiments. The operations performed by the intra-oral imaging system 104 may be caused by intra-oral imaging application 132 executing on the processor 126 of the intra-oral imaging system 104.

Control starts at block 702 in which the processor 126 of the intra-oral imaging system 104 performs operations to send data corresponding to an image of at least one tooth acquired by the intra-oral imaging sensor 134 to be displayed on the head mounted display system 102. The processor 126 receives (at block 704), from the head mounted display system 102, a selection of at least one operation that is to be performed by the processor 132. For example, the intra-oral imaging system 104 may receive an answer to a yes or no question via the nodding or shaking of the head of the operator 106, 108 analyzed via accelerator or UPS measurements.

Therefore, FIGS. 1-7 illustrate certain embodiments that show how a head mounted display system 102 may be used in association with an intra-oral imaging system 104, to facilitate the display of data and images to an operator. The head mounted display system 102 may request the intraoral imaging system 104 to perform certain operations, and the intra-oral imaging system 104 may request the head mounted display system 102 to perform certain operations.

### ADDITIONAL DETAILS OF EMBODIMENTS

The operations described in the figures may be implemented as a method, apparatus or computer program product using techniques to produce software, firmware, hardware, or any combination thereof. Additionally, certain embodiments may take the form of a computer program product embodied in one or more computer readable storage medium(s) having computer readable program code embodied therein.

A computer readable storage medium may include an electronic, magnetic, optical, electromagnetic, or semiconductor system, apparatus, or device, or any suitable combination of the foregoing. The computer readable storage medium may also comprise an electrical connection having one or more wires, a portable computer diskette or disk, a hard disk, a random access memory (RAM), a read-only memory (ROM), an erasable programmable read-only memory (EPROM or Flash memory), a portable compact disc read-only memory (CD-ROM), an optical storage device, a magnetic storage device, etc. A computer readable storage medium may be any tangible medium that can contain, or store a program for use by or in connection with an instruction execution system, apparatus, or device.

A computer readable signal medium includes a propagated data signal with computer readable program code embodied therein. A computer readable signal medium may be any computer readable medium that is not a computer readable storage medium and that can communicate, propagate, or transport a program for use by or in connection with an instruction execution system, apparatus, or device. The computer readable storage medium is different from the computer readable signal medium.

Computer program code for carrying out operations for aspects of the present invention may be written in any combination of one or more programming languages.

Aspects of the present invention are described below with reference to flowchart illustrations and/or block diagrams of methods, system and computer program products according to certain embodiments. At least certain operations that may have been illustrated in the figures show certain events occurring in a certain order. In alternative embodiments, certain operations may be performed in a different order, modified or removed. Additionally, operations may be added to the above described logic and still conform to the described embodiments. Further, operations described herein may occur sequentially or certain operations may be processed in parallel. Yet further, operations may be performed by a single processing unit or by distributed processing units. Computer program instructions can implement the blocks of the flowchart. These computer program instructions may be provided to a processor of a computer for execution.

FIG. 8 illustrates a block diagram that shows certain elements that may be included in a system 800 comprising a computational device, where the computational device may be the head mounted display system 102 or the intra-oral imaging system 104, in accordance with certain embodiments. The system 800 may include a circuitry 802 that may in certain embodiments include at least a processor 804. The processor 804 may comprise any suitable processor known in the art, such as, an arithmetic logical unit, a central processing unit, a circuitry that perform operations, hardware that performs instructions of a computer program, a microprocessor, a parallel processor, an array processor, a vector processor, a transistorized central processing unit, a microcontroller, a logic circuitry, etc. Any device that manipulates digital information based on one or more operational instructions or in a predefined manner is an example of the processor 804. The system 800 may also include a memory 806 (e.g., a volatile memory device), and storage 808. The storage 808 may include a non-volatile memory device (e.g., EEPROM, ROM, PROM, RAM, DRAM, SRAM, flash, firmware, programmable logic, etc.), magnetic disk drive, optical disk drive, tape drive, etc. The storage 808 may comprise an internal storage device, an attached storage device and/or a network accessible storage device. The system 800 may include a program logic 810 including code 812 that may be loaded into the memory 806 and executed by the processor 804 or circuitry 802. In certain embodiments, the program logic 810 including code 812 may be stored in the storage 808. In certain other embodiments, the program logic 810 may be implemented in the circuitry 802. Therefore, while FIG. 8 shows the program logic 810 separately from the other elements, the program logic 810 may be implemented in the memory 806 and/or the circuitry 802.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A system in communication with an intra-oral imaging system, the system comprising:
a processor; and
at least one head mounted display unit coupled to the processor, wherein the at least one head mounted display unit is configured to display data received from the intra-oral imaging system.

2. The system of claim 1, wherein the system comprises a head mounted display system, the system further comprising:
a measurement device coupled to the processor, wherein data from the measurement device is processed by the processor to determine movement of a head of a wearer of the head mounted display system, and wherein the movement of the head is used to select at least one operation performed by the intra-oral imaging system.

3. The system of claim 2, wherein the measurement device is selected from a group consisting of an accelerometer and a global positioning system receiver.

4. The system of claim 1, wherein the at least one head mounted display unit is selected from a plurality of head mounted display units, and wherein the plurality of head mounted display units is comprised of:
a first head mounted display unit that displays a surface imagery of a tooth received from the intra-oral imaging system to a left eye of a wearer of the head mounted display system; and
a second head mounted display unit that displays the surface imagery of the tooth received from the intra-oral imaging system to a right eye of the wearer of the head mounted display system.

5. The system of claim 1, wherein:
the system is configured to receive control instructions from a wand of the intra oral imaging system; and
the at least one head mounted display unit displays an image acquisition window displayed in a display unit included in the intra-oral imaging system.

6. The system of claim 1, wherein:
the at least one head mounted display unit is a see-through display unit that allows a patient's environment to be viewed simultaneously with the data acquired via the intra-oral imaging system.

7. The system of claim 1, wherein the displayed data corresponds to an image acquired by the intra-oral imaging system, the system further comprising:
a contrast setting to modify a contrast of the image received from the intra-oral imaging system; and
a battery to provide power to the system.

8. A method for communicating with an intra-oral imaging system, the method comprising:
communicating, via a head mounted display system, with the intra-oral imaging system; and
displaying, in at least one head mounted display unit of the head mounted display system, data received from the intra-oral imaging system.

9. The method of claim 8, the method further comprising:
processing, data obtained from a measurement device, to determine movement of a head of a wearer of the head mounted display unit; and
selecting at least one operation performed by the intra-oral imaging system, based on the determined head movement.

10. The method of claim 9, wherein the measurement device is selected from a group consisting of an accelerometer and a global positioning system receiver.

11. The method of claim 8, wherein the at least one head mounted display unit is selected from a plurality of head mounted display units, and wherein the plurality of head mounted display units is comprised of a first head mounted display unit and a second head mounted display unit, the method further comprising:
displaying, in the first head mounted display, a surface imagery of a tooth received from the intra-oral imaging system, to a left eye of a wearer of the head mounted display system; and
displaying, in the second head mounted display, the surface imagery of the tooth received from the intra-oral imaging system to a right eye of the wearer of the head mounted display system.

12. The method of claim 8, the method further comprising:
receiving, by the head mounted display system, control instructions from a wand of the intra oral imaging system; and
displaying, in the at least one head mounted display unit, an image acquisition window displayed in a display unit included in the intra-oral imaging system.

13. The method of claim 8, wherein the at least one head mounted display unit is a see-through display unit that allows a patient's environment to be viewed simultaneously with the data acquired via the intra-oral imaging system.

14. The method of claim 8, wherein the displayed data corresponds to an image received from the intra-oral imaging system, the method further comprising:
regulating a contrast setting to modify a contrast of the image received from the intra-oral imaging system; and
providing power to the intra-oral imaging system via a battery.

15. A computer readable storage medium wherein code embodied in the computer readable storage medium when executed by a processor performs operations in a head mounted display system configurable to be in communication with an intra-oral imaging system, the operations comprising:
communicating, via the head mounted display system, with the intra-oral imaging system; and
displaying, in at least one head mounted display unit of the head mounted display system, data received from the intra-oral imaging system.

16. The computer readable storage medium of claim 15, the operations further comprising:
processing, data obtained from a measurement device, to determine movement of a head of a wearer of the head mounted display unit; and
selecting at least one operation performed by the intra-oral imaging system, based on the determined head movement.

17. The computer readable storage medium of claim 16, wherein the measurement device is selected from a group consisting of an accelerometer and a global positioning system receiver.

18. The computer readable storage medium of claim 15, wherein the at least one head mounted display unit is selected from a plurality of head mounted display units, and wherein the plurality of head mounted display units is comprised of a first head mounted display unit and a second head mounted display unit, the operations further comprising:
displaying, in the first head mounted display, a surface imagery of a tooth received from the intra-oral imaging system, to a left eye of a wearer of the head mounted display system; and
displaying, in the second head mounted display, the surface imagery of the tooth received from the intra-oral imaging system to a right eye of the wearer of the head mounted display system.

19. The computer readable storage medium of claim 15, the operations further comprising:
receiving, by the head mounted display system, control instructions from a wand of the intra oral imaging system; and
displaying, in the at least one head mounted display unit, an image acquisition window displayed in a display unit included in the intra-oral imaging system.

20. The computer readable storage medium of claim 15, wherein the at least one head mounted display unit is a see-through display unit that allows a patient's environment to be viewed simultaneously with the data acquired via the intra-oral imaging system.

21. The computer readable storage medium of claim 15, wherein the displayed data corresponds to an image received from the intra-oral imaging system, the operations further comprising:
regulating a contrast setting to modify a contrast of the image received from the intra-oral imaging system; and
providing power to the intra-oral imaging system via a battery.

22. A system in communication with a head mounted display system, the system comprising:
an intra-oral imaging sensor; and
a processor coupled to the intra-oral imaging sensor, wherein the processor performs operations, the operations comprising:
sending data corresponding to an image of at least one tooth acquired by the intra-oral imaging sensor to be displayed on the head mounted display system; and
receiving, from the head mounted display system, a selection of at least one operation that is to be performed by the processor.
